(19) United States
(12) Patent Application Publication
(10) Pub. No.: US 2024/0408066 A1
(43) Pub. Date: Dec. 12, 2024

(54) Treatment Of Conditions Associated With Stress Granule Formation

(71) Applicant: The Trustees of Columbia University in the City of New York, New York, NY (US)

(72) Inventors: Joseph B Rayman, New York, NY (US); Donald W Landry, New York, NY (US); Shi-Xian Deng, White Plains, NY (US)

(21) Appl. No.: 18/784,131

(22) Filed: Jul. 25, 2024

Related U.S. Application Data
(63) Continuation of application No. PCT/US2023/061236, filed on Jan. 25, 2023.
(60) Provisional application No. 63/303,089, filed on Jan. 26, 2022.

(51) Int. Cl. A61K 31/41; A61K 31/44; A61K 31/519; A61K 31/69; G01N 33/68

(52) U.S. Cl. CPC A61K 31/41; A61K 31/44; A61K 31/519; A61K 31/69; G01N 33/6893

(57) ABSTRACT

Disclosed herein are methods of treating and/or prophylaxis of a disease or condition associated with cellular stress granule formation in a mammal via administration of small molecule compounds. Also disclosed herein are chemical compounds effective in treating diseases or conditions associated with aberrant cellular stress responses and/or stress granule formation.

### CROSS-REFERENCE TO RELATED APPLICATIONS

This application is a continuation of International Application Number PCT/US2023/061236, filed on Jan. 25, 2023, which claims priority to U.S. Provisional Application No. 63/303,089, filed on Jan. 26, 2022, the disclosures of each of which are hereby incorporated by reference in their entireties.

### TECHNICAL FIELD

Disclosed herein are methods, compounds, and compositions for managing cellular stress response.

### BACKGROUND

TIA1 is an RNA binding protein that plays a critical role in the cellular stress response. When cells are exposed to various environmental insults (e.g., oxidative stress, viral infection, etc.), TIA1 promotes the assembly of stress granules (SGs), which are cytoplasmic foci that act as temporary storage repositories for RNAs that are not required during the stress response. These RNAs are maintained in a translationally arrested state when localized to SGs, but can be returned to the polysome pool upon resolution of environmental challenge and disassembly of SGs. In addition, recruitment of signaling proteins into SGs alters cellular signaling, in particular apoptosis and cell survival. Taken together, these and other functions of SGs facilitate adaptive reprogramming of the proteome during cellular stress.

The functional activity of major SG components such as TIA1 is partly derived from an inherent propensity to form aggregated structures under physiological conditions. Multimerization of TIA1 and its recruitment into SGs is triggered by stress-dependent release of intracellular zinc, which acts as a physiological second messenger to promote reversible phase separation of TIA1 to drive SG formation (Rayman et al., 2018). Formation of SGs and other membraneless organelles is governed by phase separation.

Phase separation is a general biophysical mechanism that gives rise to membraneless organelles under appropriate physiological conditions. However, the propensity for TIA1 and other SG component proteins to undergo this type of physiological aggregation can be co-opted by a number of pathophysiological processes. Thus, it has been hypothesized that SGs can evolve into, or promote the seeding of, persistent aggregates that are cytotoxic. For example, TIA1 and SGs have been implicated in several neurodegenerative conditions, including amyotrophic lateral sclerosis (ALS), frontotemporal dementia (FTD), Alzheimer's disease (AD), and tauopathies (Maziuk et al., 2017; Mackenzie et al., 2017; Vanderweyde et al., 2016; Vanderweyde et al., 2012; Apicco et al., 2018). In addition, mutations in TIA1 itself are associated with Welandar distal myopathy (WDM) (Hackman et al., 2013; Klar et al., 2013). A hallmark of these disorders is the presence of pathological protein inclusions that are often positive for SG components such as TIA1, TDP-43, and Tau. In addition, there is evidence that TIA1 can drive the formation of toxic tau oligomers independent of SGs (Ash et al., 2021, PNAS). Together, these results and those of other studies suggest that inhibition of TIA1 activity and/or SG formation may represent a useful therapeutic approach for treating tauopathies and other neurodegenerative conditions associated with persistent protein aggregation (Ash et al., 2020; Fang et al., 2019; Apicco et al., 2018; Berger et al., 2007; Cowan et al., 2013; Jouanne et al., 2017; Jiang et al., 2019; Fernandes et al., 2018; Brettschneider et al., 2014; Hergesheimer et al., 2019; Neumann et al., 2006; Protter & Parker, 2016; Wolozin & Ivanov, 2019).

The functional connection between TIA1 and neurodegeneration is thought to arise from its tendency to form protein aggregates that may interact with other aggregation prone-proteins involved in these neurodegenerative processes. With respect to tau-mediated neurodegeneration, which is particularly relevant to AD and FTD, a number of compelling studies have linked TIA1, SG formation, and tau pathology. For example, interaction of TIA1 and tau regulates tau pathophysiology by modulating the generation of toxic tau oligomers Vanderweyde et al., 2016; Jiang et al., Acta Neuropath. 2019; Ash et al., PNAS 2021). Importantly, the cognitive deficits associated with overexpression of humanized tau in mice are reversed when the mice are crossed into a TIA1-deficient background (Apicco et al., 2018). Furthermore, kinase inhibitors that act upstream of TIA1-dependent SG formation ameliorate neurodegeneration (Vanderweyde et al., 2016). Similarly, TIA1 functionally interacts with TDP-43, a key splicing factor that becomes pathologically localized to SG-like aggregates in ALS/FTD, while pharmacological manipulations that impair TDP-43+ SG formation may ameliorate cellular pathological changes (Fang et al., 2019).

### SUMMARY

Disclosed herein are methods of treating and/or prophylaxis of a disease or condition associated with cellular stress granule formation in a mammal, the method comprising administering one or more of the compounds of Formulas (I) to (IV), or a pharmaceutically acceptable salt thereof:

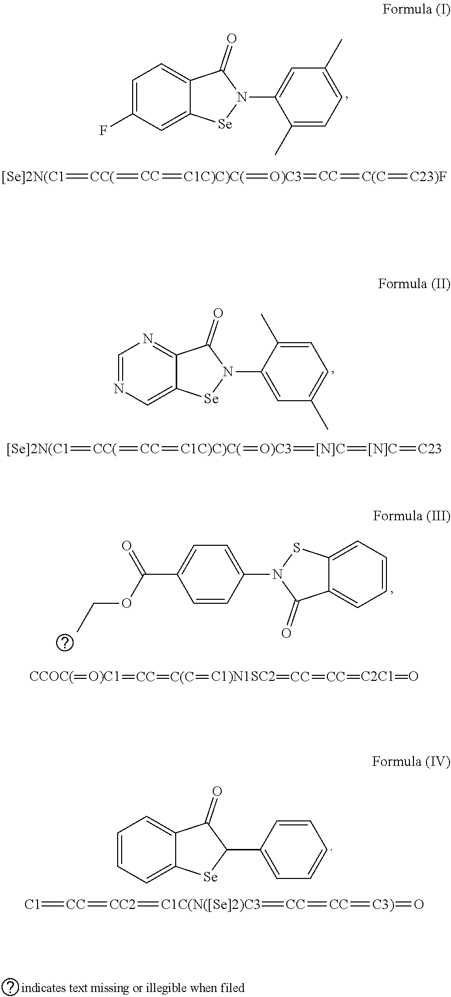

Disclosed herein are compounds according to any one of Formula (I), Formula (II), or a pharmaceutically acceptable salt thereof:

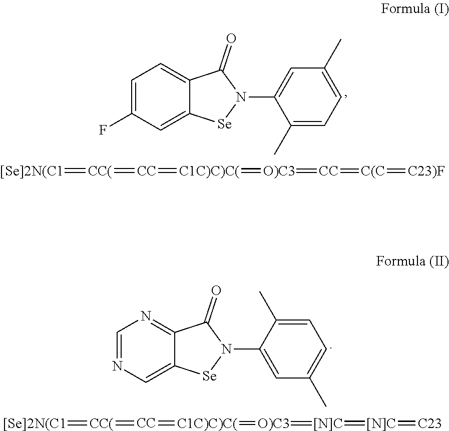

Disclosed herein are compositions comprising one or more compounds according to Formula (I), Formula (II), or a pharmaceutically acceptable salt, solvate, hydrate or tautomer thereof:

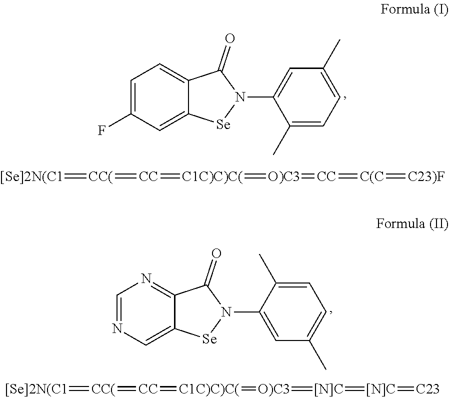

Disclosed here are methods of identifying agents that prevent and/or reduce cellular stress granule formation comprising a.) incubating a TIA1 protein or a TIA1 fusion protein with one or more agents; and b.) detecting the dimerization, oligomerization, or other multimeric state of the TIA1 protein or the TIA1 fusion protein, wherein the presence of multimeric TIA1 or multimeric TIA1 fusion protein indicates that the agent is capable of preventing and/or reducing cellular stress granule formation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the methods, compounds, and compositions disclosed, there are shown in the drawings exemplary embodiments; however, the methods, compounds, and compositions disclosed are not limited to the specific methods, compounds, and compositions disclosed. In addition, the drawings are not necessarily drawn to scale. In the drawings:

FIG. 1 illustrates an exemplary Förster resonance energy transfer (FRET) assay capable of identifying drugs that effect TIA1 multimerization. The assay can detect multimerization of TIA1 protein. When the assay is performed in the presence of other agents, such as small molecules, their effect on TIA1 multimerization can be evaluated.

FIG. 2 illustrates an exemplary method for the in vivo treatment of a disease associated with stress granule formation with a TIA1 antagonist. Hippocampal cross sections are shown in a normal mouse, a PS19 mouse, and a PS19 mouse treated with a TIA1 antagonist. Treatment of a PS19 mouse with a TIA1 antagonist inhibits synapse degeneration normally observed in these mice.

FIG. 3 illustrates two chemical synthesis processes used to create the TIA1 antagonists claimed herein. Active, commercially available compounds were either obtained from commercial sources or synthesized. Reaction scheme 1 was used to synthesize OTX1017 (Formula I in claims) and OTX1018 (Formula II in claims).

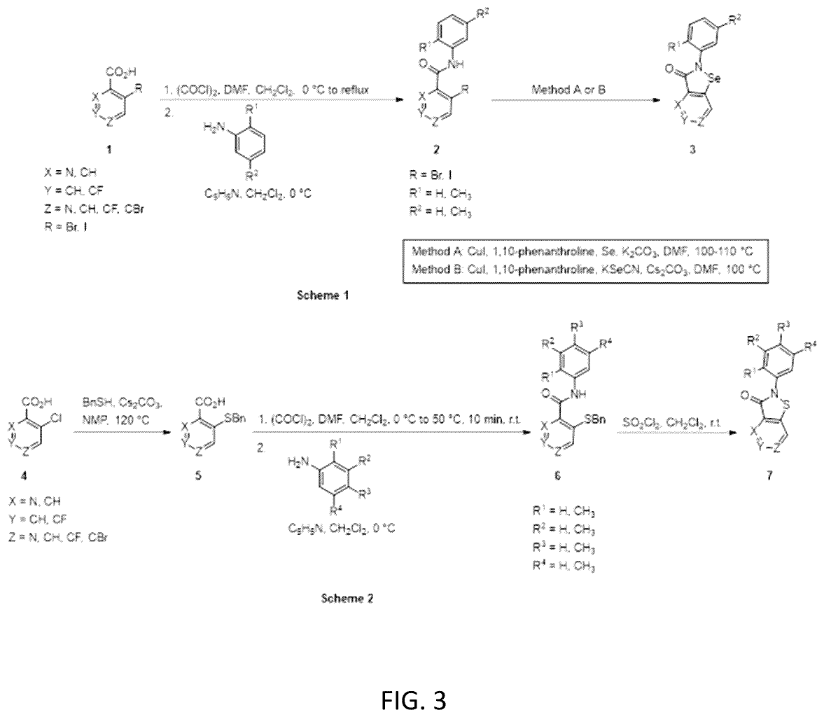

FIG. 4 illustrates the activity of novel small compounds and known compounds in driving multimerization of TIA1 via a FRET assay. An inactive compound would exhibit a flat line at 100% across the concentration range (not shown). Ref. 49 is shown as a positive control (top panels). The graphs demonstrate that OTX1017 (Formula I in claims), OTX1018 (Formula II in claims), and Formula III, and Formula IV drive multimerization of TIA1.

FIG. 5A and FIG. 5B illustrate the effect of TIA1 antagonists on the ability of TIA1 to promote tau oligomerization in vitro. FIG. 5A shows immunoblotting of recombinant TIA1:tau with different primary antibodies that recognize oligomeric tau (oTau) or TIA1 in the presence or absence of a TIA1 antagonist. Quantification of signal from the immunoblot is shown in the bar graph. FIG. 5B shows immunoblotting of recombinant TIA1:tau with anti-oTau antibodies at different concentrations of the TIA1 antagonists OTX0049 and OTX1017 (Formula I in claims) at different time points. Quantification of signal from the immunoblot after 1 hour is shown in the graph. Compounds shown to be TIA1 antagonists interfere with tau oligomerization.

FIG. 6 illustrates the effect of a TIA1 antagonist treatment on tau:microtubule interaction in PS19 tauopathy mice. PS19 mice were treated with a TIA1 antagonist and hippocampal tissue was harvested with microtubule-associated and microtubule-non associated extracts were immunoblotted with anti-Tau5 antibody. Quantification of the ratio of microtubule bound tau versus soluble tau for is shown in the bar graph for treated and control mice. Chronic treatment of PS19 mice with a TIA1 antagonist produces an increased ratio of microtubule-bound vs. unbound tau, approximating what is observed when TIA1 is deleted in the PS19 mice.

FIG. 7 illustrates an exemplary method directed to treatment of tauopathies. Tg4510 transgenic mice were treated with a TIA1 antagonist. Hippocampal slices were then prepared for conventional immunohistochemistry with GFAP (neuroinflammation marker) and NeuN (neuronal marker). Quantification of GFAP and NeuN straining is shown in the dot plots. TIA1 antagonist reduces neuroinflammation and increases neuronal number and cell body volume in a well-established model of tauopathy.

FIG. 8A, FIG. 8B, and FIG. 8C illustrate the effect of TIA1 antagonists on arsenite-induced stress granule formation in cells. Sodium arsenite consistently induces stress granule formation in virtually all cells, while treatment with a TIA1 antagonist inhibits arsenite-induced stress granule formation. In FIG. 8A, arrows indicate endogenous TIA1- and tau-positive stress granules. FIG. 8B shows that TIA1 antagonists reduce the number of sodium arsenite induced stress granules per cell. FIG. 8C shows that TIA1 antagonists reduce the average size of arsenite-induced tau foci.

FIG. 9 illustrates the effect of TIA1 antagonists on puromycin induced stress granules in human motor neurons. The fluorescence micrographs shows that co-incubation of puromycin with OTX0049 at indicated concentrations completely inhibits stress granule formation. The graph shows that the drug-dependent inhibition of TIA1-positive stress granules is evident across a variety of human ALS patient-derived motor neuron lines.

FIG. 10 illustrates the inhibition of tau oligomerization in vivo by OTX1017. Mice were treated with 10 mg/kg OTX1017 i.p., once per week, for 6 weeks, hippocampal extracts were prepared, and dot blots were performed with an oligomeric tau-specific antibody. These results demonstrate that treating PS19 transgenic mice with OTX1017 significantly reduces the presence of oligomeric tau in the hippocampus.

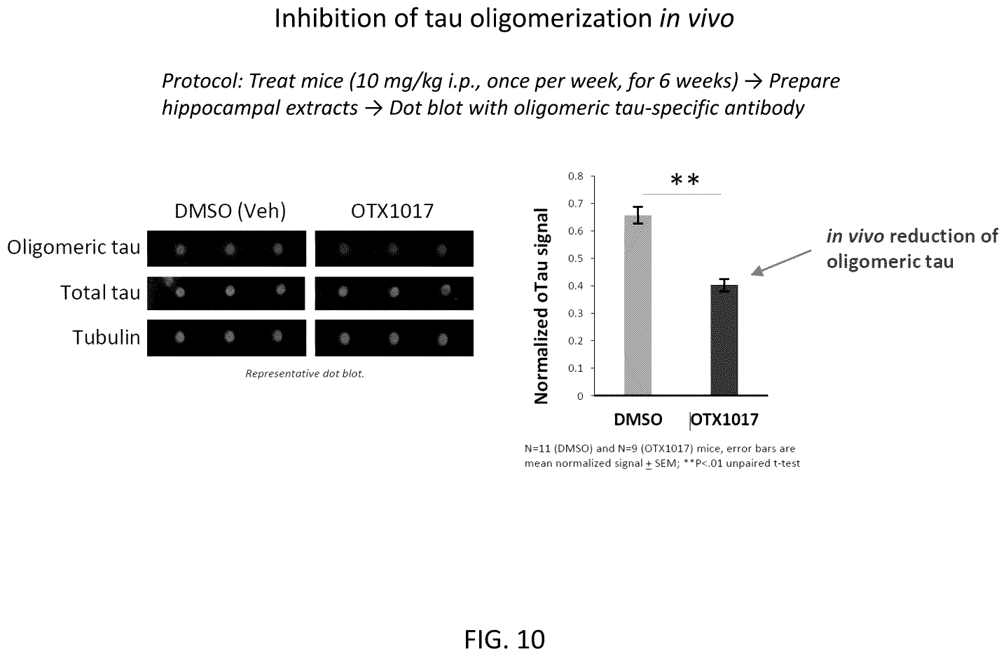

### DETAILED DESCRIPTION OF ILLUSTRATIVE EMBODIMENTS

The disclosed methods, compounds, and composition may be understood more readily by reference to the following detailed description taken in connection with the accompanying figures and examples, which form a part of this disclosure. It is to be understood that the disclosed methods, compounds, and compositions are not limited to the specific methods, compounds, and compositions disclosed, described, and/or shown herein, and that the terminology used herein is for the purpose of describing particular embodiments by way of example only and is not intended to be limiting of the claimed methods, compounds, and compositions disclosed. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the methods, compounds, and compositions disclosed which are, for clarity, described herein in the context of separate embodiments, may also be provided in combination in a single embodiment. Conversely, various features of the methods, compounds, and compositions that are, for brevity, described in the context of a single embodiment, may also be provided separately or in any subcombination. Further, reference to values stated in ranges include each and every value within that range.

Disclosed herein are methods for treating and/or prophylaxis of a disease or condition associated with cellular stress granule formation in a mammal. Mammals may include, but are not limited to humans. The mammal treated by the disclosed methods may be a human. Those in the art will appreciate that certain veterinary diseases and conditions are also associated with stress granule formation and could be applications of the present methods, compounds, and compositions.

The method can comprise treating and/or prophylaxis of a disease or condition associated with cellular stress granule formation in a mammal, the method comprising administering one or more of the compounds of Formulas (I) to (IV), or a pharmaceutically acceptable salt thereof:

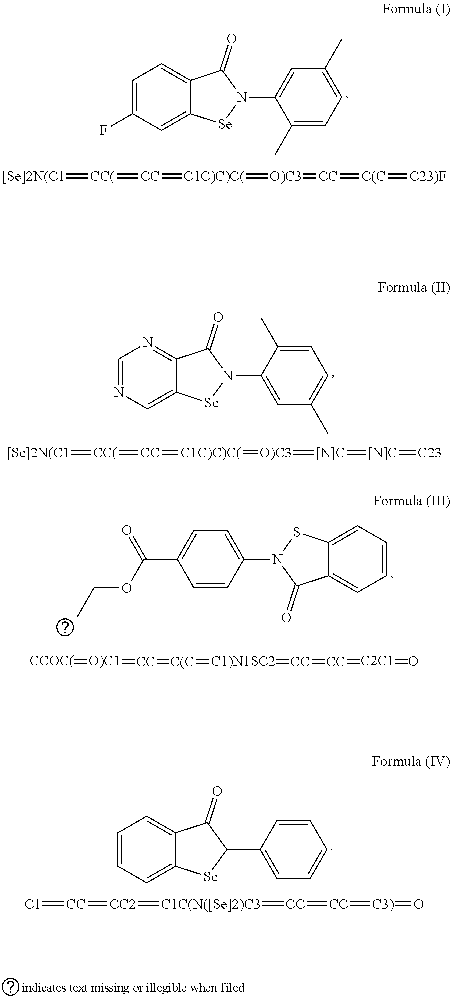

The cellular stress granules can comprise TIA1, Tau, TDP-43, or a combination thereof. In some embodiments, the cellular stress granules comprise TIA1. In some embodiments, the cellular stress granules comprise Tau. In some embodiments, the cellular stress granules comprise TDP-43.

Administration of the one or more compounds can promote multimerization of TIA1. Administration of the one or more compounds can prevent or decrease cellular stress granules or the formation thereof. Administration of the one or more compounds can prevent or decrease Tau or TDP-43 aggregation or oligomerization.

Treatment used herein describes the amelioration of a sign, symptom, undesirable effect, or underling cause of a disease or condition. Prophylaxis as used herein describes the protection against, prevention, or delay in onset of a disease or condition.

Diseases, conditions, or disorders as used herein refer to a recognized deviation from the normal structural or functional state of an organism. Diseases to be treated can be of any type, including but not limited to, neurological, cancerous, autoimmune, congenital, and infectious. The disease or condition can be a neurodegenerative disorder, a viral infection, a disease-linked genetic mutation, Welander distal myopathy, chronic stress, aging, psychiatric illness, or cancer. The neurodegenerative disorders can be amyotrophic lateral sclerosis (ALS), frontotemporal dementia (FTD), Alzheimer's disease (AD), and tauopathies. The psychiatric illness can be post-traumatic stress disorder (PTSD) or anxiety. The cancers to be treated can be those associated with a KRAS mutation.

The disclosed compounds can be administered for a known disease or condition recognized to be associated with stress granules. However, the diseases and conditions contemplated for treatment are circumscribed by an association with stress granule formation rather than any limitation based on the type of disease or condition. The diseases or conditions associated with stress granule formation are not limited to those which have so far been identified as being associated with stress granule formation as different biological diseases or conditions may ultimately prove to be associated with stress granule formation. Disclosed herein are methods to prevent and/or reduce the formation of stress granules and this method is applicable to any disease or condition of the body that could benefit from the prevention and/or reduction of stress granules regardless of whether such a stress granule association is presently known.

In addition to familiar/recognized diseases, conditions as used herein include other states of the body which have or cause prejudicial and/or undesirable effects. Conditions to be treated can be of any type, including but not limited to aging, anxiety, or stress. Conditions to be treated need not be a recognized disease state.

A disease or condition associated with cellular stress granule formation as used herein includes any disease state or condition of the body in which cellular stress granules are formed as a sign, symptom, effect, or underlying cause. This includes all diseases or conditions that are presently known to be associated with stress granule formation or those diseases or conditions that are discovered to be associated with stress granule formation in the future.

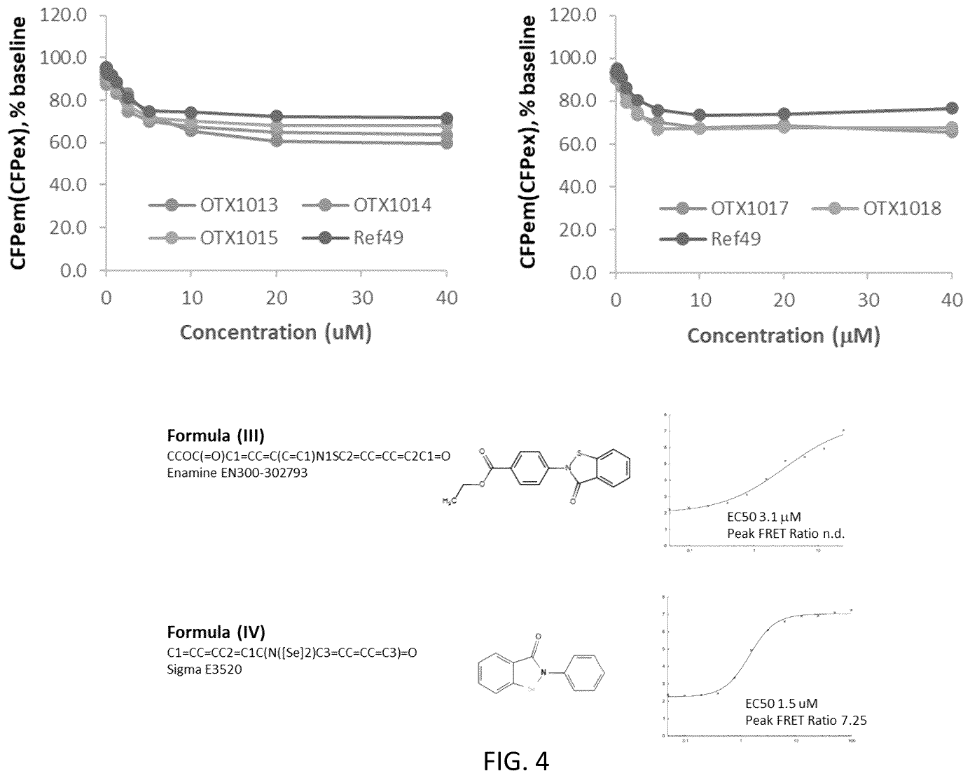

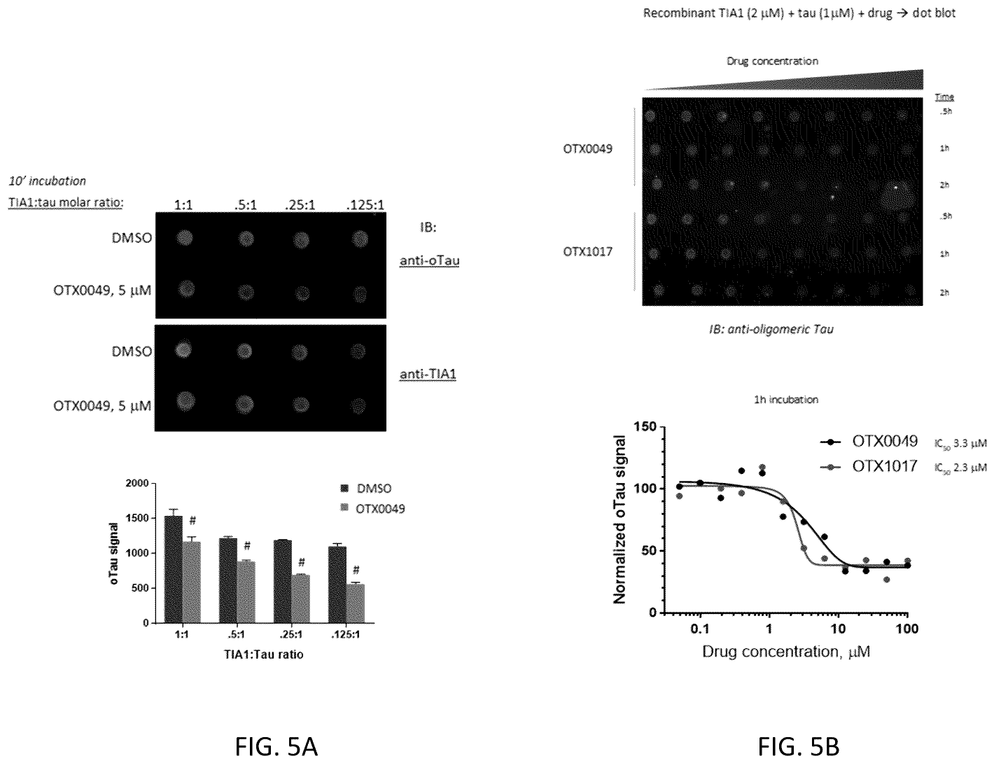

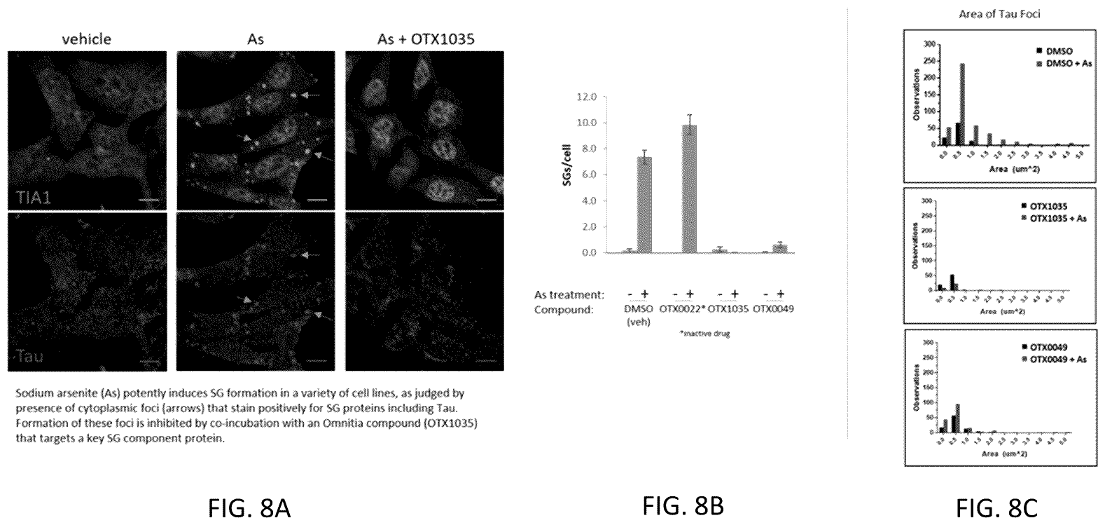

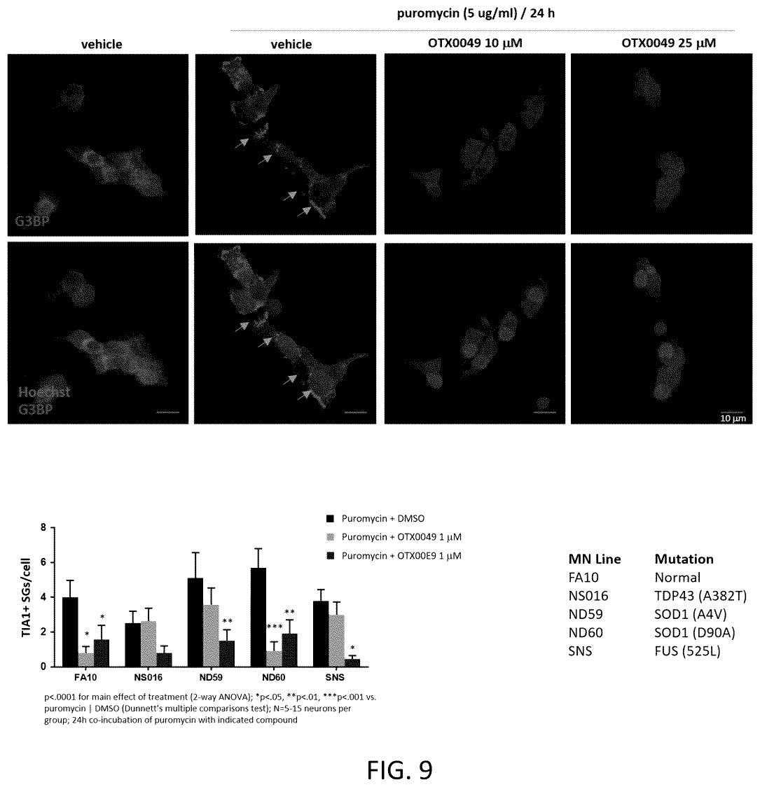

“Stress granules” as used herein refers to cytoplasmic foci that form primarily as part of the cellular stress response. The stress granules can comprise components that include, but are not limited to, RNA, TIA1, TDP-43, and Tau. The compounds described herein show activity toward TIA1, however, TIA1 inclusion in the stress granule is not required for effective treatment of a disease or condition associated with stress granule formation. As FIG. 5 demonstrates, multimerization of tau protein can be reduced by compounds that antagonize TIA1. Further, the compounds described herein block the formation of stress granules in cellular assays and also inhibit TIA1-dependent oligomerization of tau in vitro despite causing TIA1 to multimerize (FIG. 4, FIG. 5, and FIG. 8). Nevertheless, the compounds described herein influence TIA1 activity and enforce a multimeric TIA1 configuration that is 1) incompatible with stress granule formation and 2) ineffective in promoting tau oligomerization (FIG. 4, FIG. 8, and FIG. 9). Because TIA1 is a key nucleator of stress granules, the compounds described herein could therefore be compatible with preventing and/or reducing the formation of stress granules despite their compositional heterogeneity.

The compound or pharmaceutically acceptable salt thereof administered to the mammal can comprise that of:

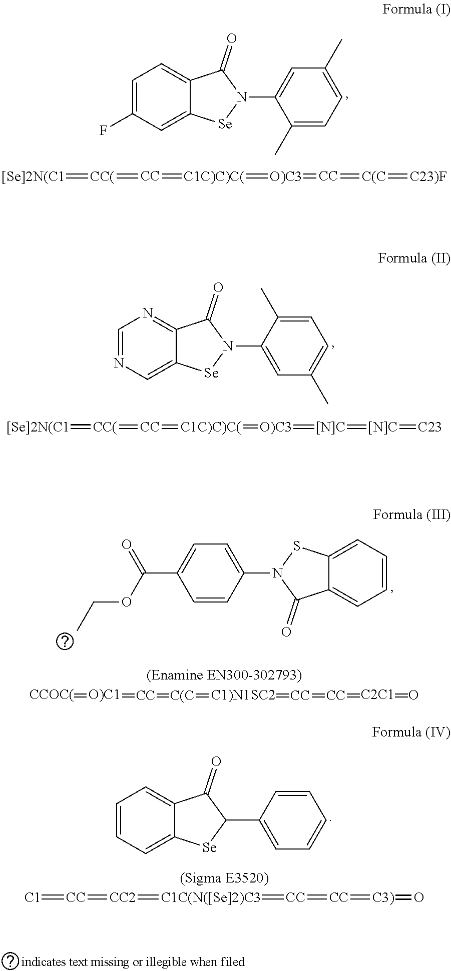

or combinations thereof.

The compound or compound combination administered to the mammal can be performed via any route known in the art, including but not limited to intravenous, oral, intramuscular, or subcutaneous. Those of skill in the art will appreciate that methods of administration can be diverse and dependent on the underlying disease or condition that is to be treated.

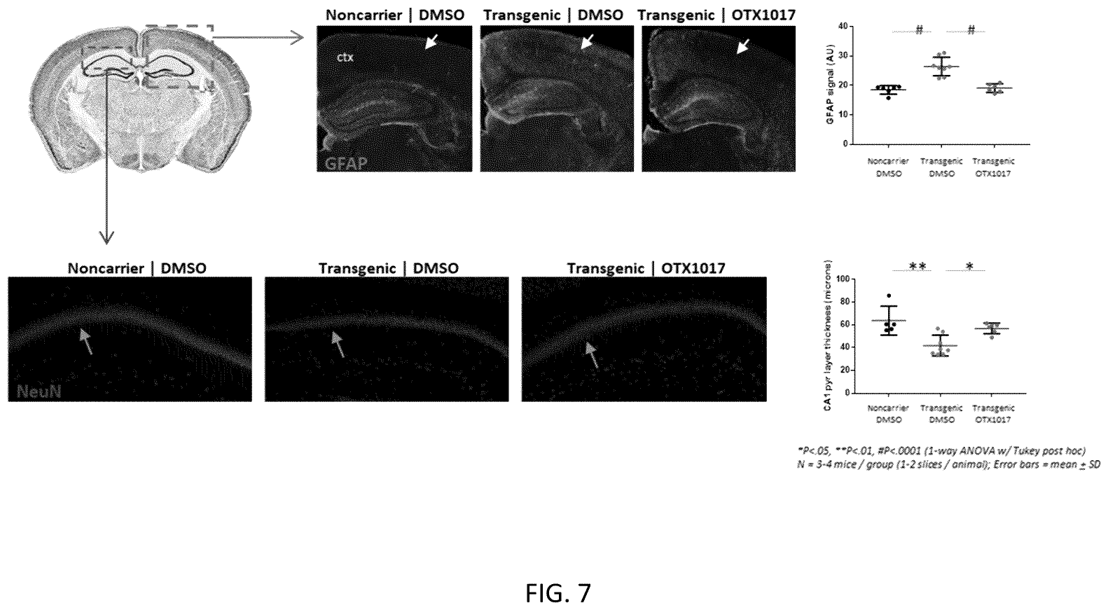

The compounds described herein were identified through their ability to multimerize TIA1 in vitro (FIG. 4). These compounds prevent and/or reduce the formation of stress granules or the effects and pathologies thereof (FIG. 7, FIG. 8, and FIG. 9). The compounds can promote the multimerization of TIA1 in vivo as part of their mechanism of action. However, given the complexity of stress granule formation and their multitude of components, it is also possible that the compounds have other diverse effects on TIA1 in vivo not limited to TIA1 multimerization that nonetheless prevent and/or reduce stress granule formation.

The compounds described herein can be administered to prevent stress granules before they form. In subjects diagnosed with or having predispositions to diseases or conditions associated with stress granule formation, the compounds described herein may be administered prior to stress granule formation in order to prevent their formation. The compounds described herein can be administered to reduce, maintain, or eliminate stress granules that have already formed in association with a disease or condition. Those in the art will appreciate that different diseases or conditions will be characterized by different stress granule pathologies which will be associated with distinct symptoms. Treatment with the compounds described herein may slow the formation of stress granules. Treatment with the compounds described herein may maintain the stress granules that have already formed. Treatment with the compounds described herein may reduce or eliminate the stress granules that have already formed.

Administration of the compound(s) can prevent and/or decrease Tau or TDP-43 aggregation or oligomerization. Tau and TDP-43 represent common components of stress granules known to those in the art. However, stress granules may also comprise diverse components, the oligomerization and association of which could be influenced by TIA1, and consequently affected by the compounds described herein.

Administration of the compound(s) described herein can be performed in combination with a chemotherapeutic drug where desirable. The herein disclosed methods can further comprise administering chemotherapeutic drugs to the mammal. As some cancers are associated with stress granule formation, a combination of the compounds described herein and a chemotherapeutic agent may enhance treatment of certain cancers. The chemotherapeutic drug can be sorafenib or bortezomib.

Within the disclosed methods, following the administering the compound can be delivered to a neuron. Many of the diseases currently known to be associated with stress granule formation are neurological in nature and result in stress granule formation within neurons. To exert a therapeutic effect in these diseases, the compounds can be delivered to a neuron to exert their therapeutic effects.

Disclosed herein are compounds according to any one of Formula (I), Formula (II), or a pharmaceutically acceptable salt thereof:

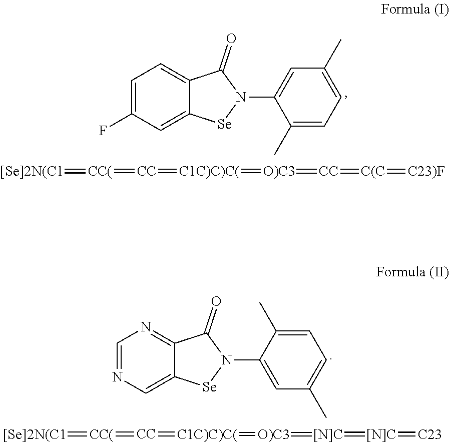

Disclosed herein are compositions, comprising one or more compounds according to Formula (I), Formula (II), or a pharmaceutically acceptable salt, solvate, hydrate or tautomer thereof:

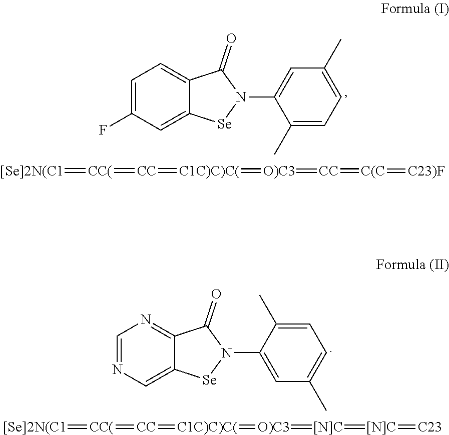

Disclosed herein are methods of identifying agents that prevent and/or reduce cellular stress granule formation. The methods of identifying agents that prevent and/or reduce cellular stress granule formation can comprise incubating a TIA1 protein or a TIA1 fusion protein with one or more agents; and detecting the dimerization, oligomerization, or other multimeric state of the TIA1 protein or the TIA1 fusion protein, wherein the presence of multimeric TIA1 or multimeric TIA1 fusion protein indicates that the agent is capable of preventing and/or reducing cellular stress granule formation.

Agents that promote multimeric states of TIA1 in vitro can reduce stress granule formation in vivo (FIG. 4, FIG. 8, and FIG. 9). As a result, agents that prevent and/or reduce stress granule formation can be identified in vitro by their ability to promote the multimeric TIA1 state. The presence of multimeric TIA1 can be detected via Förster resonance energy transfer (FRET). As used herein, multimeric state refers to a protein state that comprises one or more TIA1 proteins in an associated state, including but not limited to a dimer, trimer, or oligomer. An agent can be a small molecule compound or a macromolecule, including but not limited to proteins, nucleic acids, or lipids. In some embodiments, the agent is a small molecule. Further, a composition comprising a combination of agents can be assessed.

As used herein, a TIA1 protein means mammalian copies of expressed TIA1 protein. A TIA1 fusion protein can comprise a TIA1 component linked to another proteinaceous component. The other proteinaceous component can be any protein, but preferably one that aids detection of a TIA1 multimeric state. The fusion protein can comprise TIA1 and a fluorescent protein. Those in the art are aware of a multitude of proteinaceous components that can comprise a fusion protein and facilitate detection via various processes including but not limited to fluorescence, luminescence, or enzymatic activity.

Incubation as used herein comprises combination of the TIA1 protein or TIA1 fusion protein with the agent or combinations thereof to be assessed for the ability to promote a multimeric TIA1 state such that the agent is capable of exerting its effects on TIA1. Detecting a multimeric state of TIA1 can be performed by any method known in the art including but not limited to Förster resonance energy transfer (FRET), microscopy, or immunoblotting, or electrophoresis.

### EXAMPLES

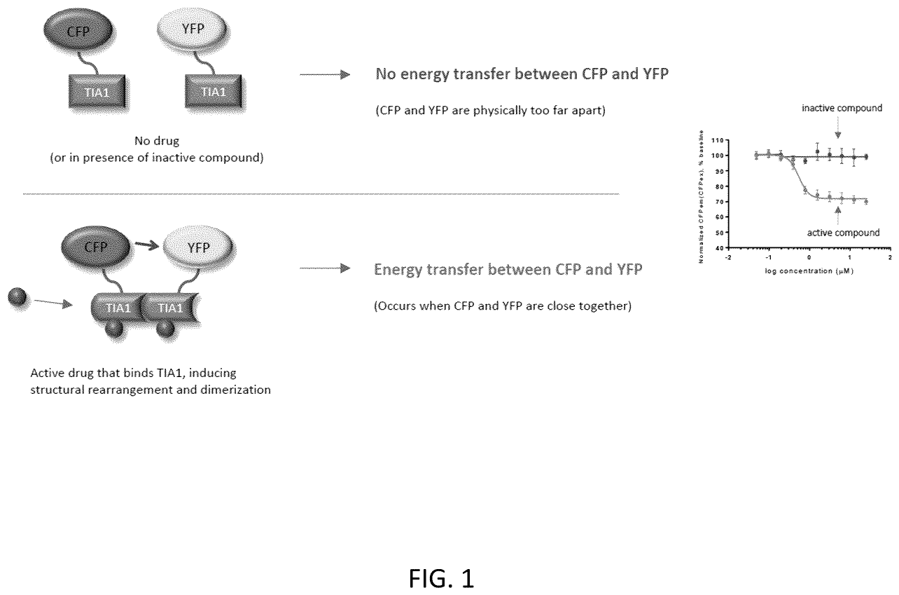

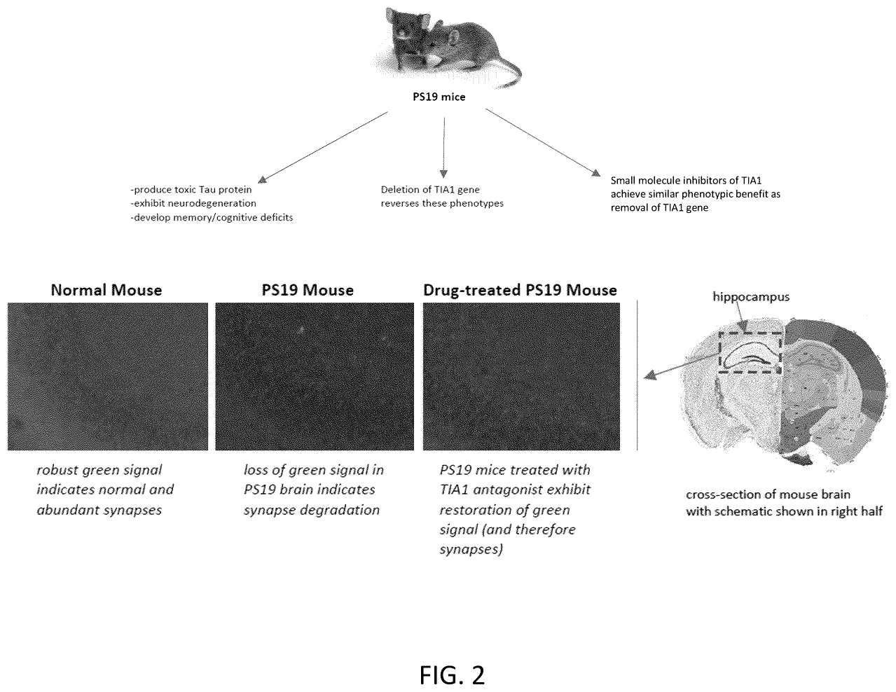

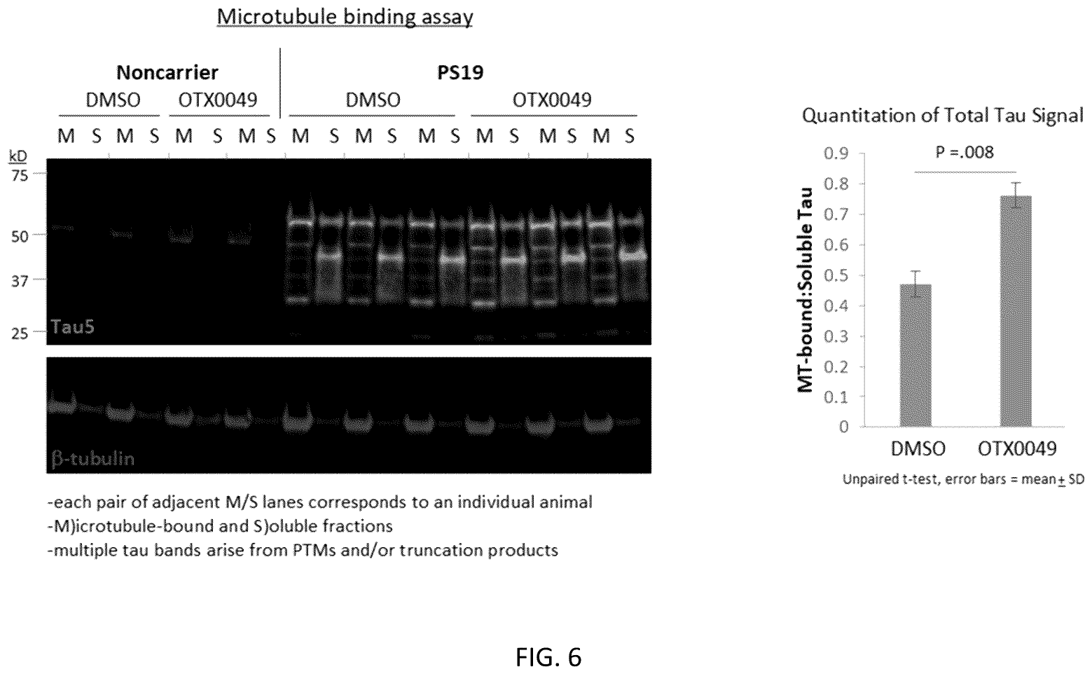

FIG. 1 demonstrates how compounds were identified through Förster resonance energy transfer (FRET). The assay that takes advantage of a phenomenon involving the transfer of energy between two compatible fluorescent proteins. In particular, the fluorescent proteins CFP and YFP (cyan fluorescent protein and yellow fluorescent protein, respectively) emit fluorescent light of characteristic wavelengths when excited by laser radiation. CFP or YFP was tethered to a TIA1 protein and the hybrid proteins were mixed together. Subsequently, if TIA1 self-interacts (dimerizes), the tethered CFP and YFP proteins are brought into close proximity to one another. When this occurs, laser excitation of CFP leads to a release of energy that is absorbed by YFP, which can be detected in the assay. This assay can be employed in the presence of other agents, such as small molecule compounds, to determine whether their presence has any effect on dimerization of TIA1. This screen was employed to assay compounds in a chemical library in order to identify compounds that effect TIA1 binding and dimerization or multimerization. Somewhat paradoxically, small molecules that enhance TIA1 multimerization identified via positive FRET signal actually block the formation of SGs in cellular assays and also inhibit TIA1-dependent oligomerization of tau in vitro with associated phenotypic benefits in vivo (FIG. 2, FIG. 5, FIG. 6, FIG. 7, FIG. 8, and FIG. 9). Small molecules that promote a multimeric TIA1 configuration (hence FRET-positive) are nevertheless incompatible with recruitment into SGs and incapable of driving tau oligomerization.

FIG. 2 demonstrates that treatment with a compound that antagonizes TIA1 can reduce neurodegeneration. A characteristic feature in neurodegenerative disorders is the degradation or loss of synapses, the physical interfaces that connect neurons to one another. Synapse loss causes deficits in memory and cognition, and is observed in aged PS19 mice and other genetic mouse models. As PS19 mice age, they develop many Alzheimer's Disease-related phenotypes, including neurodegeneration, memory deficits, and accumulation of toxic tau species. In a PS19 mouse, genetic tools to reduce the level of TIA1 protein, such as deletion of the TIA1 gene, can reduce these phenotypes which can also be recapitulated via treatment with a TIA1 antagonist.

To visualize synapse loss in the hippocampus, a key brain area involved in learning and memory storage, the prevalence of a synaptic marker called synaptophysin, a protein that is normally abundant in synapses, can be assessed. Synaptophysin staining, shown in green in the images of mouse hippocampal tissue shown below, is reduced in PS19 mice compared to control mice that do not harbor human tau (compare 1st and 2nd panels of FIG. 2). Green signal indicates normal and abundant synapses, which are clearly identifiable in the normal mouse. In contrast, the PS19 shows a loss of green signal indicative of neurodegeneration. However, when PS19 mice are treated with a small molecule antagonist of TIA1, the green signal is largely restored (3rd panel of FIG. 2), suggesting that pharmacological inhibition of TIA1 prevents loss of synapses normally observed in PS19 mice.

### Experiment 1: In Vitro FRET Assay Demonstrating that Compounds OTX1013, OTX1014, OTX1015, OTX1017 (Formula I in Claims), and OTX1018 (Formula II in Claims) Induce Multimerization of Recombinant Mouse TIA1 (FIG. 4)

One way to establish that test compounds bind recombinant TIA1 is to perform a Förster resonance energy transfer (FRET) experiment. Binding of test compound to monomeric protein is inferred by a change in FRET signal upon ligand-induced multimerization of suitably tagged (c.g.,-ECFP and -EYFP) fusion proteins. In other words, not only must the compound bind to TIA1-ECFP and TIA1-EYFP, it must also induce a change in multimeric state such that energy can be transferred between the ECFP and EYFP tags. Alternatively, if the fusion proteins exist in a multimeric configuration in the absence of ligand (and therefore produce a baseline FRET signal), a compound that binds to the fusion proteins may disrupt multimerization and inhibit the FRET signal. However, for TIA1-ECFP and TIA1-EYFP, the proteins do not multimerize under basal conditions (not shown).

Plasmids encoding mouse TIA1-ECFP and TIA1-EYFP were generated by standard PCR-based cloning techniques. Full-length mouse TIA1 cDNA was generated from standard RT-PCR from total mouse brain RNA and cloned into pRSET bacterial expression vector to generate pRSET-TIA1-ECFP and pRSET-TIA1-EYFP, which also encode N-terminal 6× His tag. Plasmids were purified and sequenced to verify constructs. Recombinant protein was generated by standard protocols using BL21 AI competent cells and purified by standard Ni2+ chromatography. For the FRET assay, recombinant mouse TIA1-ECFP+TIA1-EYFP (10 ug/ml final concentration each) are incubated with a range of test compound concentrations (0-40 μM in DMSO) in reaction buffer (10 mM Tris-HCl, 150 mM NaCl, pH 7.5) in standard 96-well plate format. Samples are read in real-time with a fluorescence microplate reader. To measure donor FRET, samples are excited at 430+30 nm, and excitation is measured at 480+20 nm. Compounds that drive multimerization of the fusion proteins cause proximal localization of the ECFP and EYFP moieties, leading to a reduction in ECFP fluorescence with ECFP excitation due to energy transfer to EYFP. Ref49 is a positive control compound that was identified based on results from the initial high-throughput FRET screen. Compounds are not inherently fluorescent and do not directly interfere with fluorescence-based measurements. Additional relevant controls for this assay published in (Rayman et al. 2018, Cell Reports).

All of the above OTX compounds drive multimerization of TIA1 to produce comparable FRET signals which exceed the FRET change induced by the positive control, Ref. 49 (FIG. 4, representative data from at least 3 independent experiments). Known compound Formula III (Enamine EN300-302793) and Formula IV (Sigma E3520) also demonstrate FRET signals indicative of TIA1 multimerization. An inactive compound would exhibit a flat line at 100% across the concentration range (not shown).

These results indicate that the OTX compounds listed above bind to recombinant TIA1 and drive its multimerization. In summary, this assay demonstrates that the OTX compounds bind to TIA1 in vitro and cause a structural rearrangement in the protein.

### Experiment 2: Drug-Dependent Inhibition of TIA1-Induced Oligomerization of Tau Protein (FIG. 5).

Oligomeric tau is the most pathological configuration of the protein. Studies have demonstrated the TIA1 drives the oligomerization of tau, and consequently, TIA1 antagonists should interfere with the ability of TIA1 to promote tau oligomerization in vitro.

Purified full-length recombinant mouse TIA1 protein was generated from standard cloning methods followed by standard protocols for bacterial expression and purification. Recombinant TIA1 was incubated with commercially available human recombinant tau (ON4R isoform) obtained from R&D Systems at the indicated molar ratios/concentrations. After indicated incubation times, reactions were spotted onto nitrocellulose membrane followed by immunoblotting with primary antibodies that recognize oligomeric tau (oTau) or TIA1. Fluorescently labeled secondary antibody staining against indicated primary antibodies was followed by quantitative image analysis using a Li-COR Odyssey system. Experiment A evaluates the impact of TIA1:tau molar ratio on tau oligomerization and whether OTX0049 at a single test concentration interferes with TIA1-dependent tau oligomerization (FIG. 5, left panels). The experiment represented in the right panels measure the dose-response of OTX0049 and OTX1017 (Formula I in claims) inhibition of TIA1-dependent tau oligomerization at a fixed ratio of recombinant TIA1:tau (FIG. 5, right panels).

In the experiments represented in the left panels of FIG. 5, increasing the ratio of TIA1:tau increases tau oligomerization, as determined by quantitative imaging of anti-oligomeric tau (oTau) immunoblotting. This observation replicates earlier published data (Ash et al., PNAS 2021). Co-incubation with OTX0049 (Ref49) causes a further significant reduction in tau oligomerization (FIG. 5, left panels). (Quantitation is from triplicate data points followed by 2-way ANOVA with post hoc results indicated (#p<0.0001)). The experiments represented in the right panels of FIG. 5 shows drug-dependent reduction of TIA1-dependent tau oligomerization as a function of drug concentration (OTX0049 or OTX1017 (Formula I in claims)) and time (0.5-2 h), with quantitation of the 1 h incubation point shown in the graph (FIG. 5, right panels). Dose-response curves indicate that compounds exhibit similar IC50s values

Tau oligomerization in vitro is enhanced by co-incubation with TIA1. Here, compounds OTX0049 and OTX1017 (Formula I in claims) are shown to interfere with tau oligomerization. These findings are consistent with a model in which ligand binding to TIA1 enforces a structural configuration that is either not permissive for TIA1-tau interaction or inhibits productive structural rearrangement (oligomerization) of tau when bound to TIA1. These findings suggest that interfering with tau oligomerization with small molecules that target TIA1 may be a useful therapeutic strategy.

### Experiment 3: Drug-Induced Normalization of Tau:Microtubule Interaction in PS19 Tauopathy Mice (FIG. 6)

Tau is a microtubule binding protein that has been implicated in the regulation of microtubule dynamics. Furthermore, hyperphosphorylation of tau, which occurs in Alzheimer's Disease, is correlated with impaired tau function and decreased microtubule binding. In the PS19 mouse model, neurodegenerative changes in the brain are hypothesized to be driven by the accumulation of excess pools of non-microtubule-associated tau. Collective results raise the possibility that non-microtubule-associated tau is more likely to interact with cytoplasmic TIA1, thereby increasing the probability of tau oligomerization. Importantly, when PS19 mice are crossed into a TIA1-deficient background, there is phenotypic rescue of tau:microtubule interaction and amelioration of neurodegenerative phenotypes at the cellular and behavioral level. These observations raise the possibility that drug-induced inhibition of TIA1:tau interaction may restore tau binding to microtubules and concomitant phenotypic rescue. In this experiment, the impact of chronic administration of OTX0049 on tau:microtubule interaction was evaluated.

PS19 mice are treated with DMSO vehicle or OTX0049 (2 mg/kg i.p.) once per week for 8 weeks. Hippocampal tissue was harvested, and microtubule-associated and-nonassociated extracts were prepared as described (Apicco et al. 2018, Nat. Neurosci.), followed by anti-Tau5 immunoblotting. Tau signals were quantitated using NIH ImageJ.

PS19 mice overexpress a humanized form of mutant tau that is readily detectable in hippocampal extracts from transgenic mice, but not from noncarrier controls. Multiple bands are detected in the PS19 mice which correspond to a population of tau isoforms that reflect different post-translational modifications, splice isoforms, and truncations (FIG. 6). Quantitation of microtubule-bound (M) vs. soluble(S) from 3 transgenic mice per condition (DMSO vs. OTX0049) reveals a significant increase in the ratio of Tau5 signal in OTX0049-treated animals (FIG. 6).

Chronic treatment of mice with a TIA1 antagonist produces an increased ratio of microtubule-bound vs. unbound tau, approximating what is observed when TIA1 is deleted in the PS19 mice (Apicco et al. 2019, Nat. Neurosci.). These studies suggest that pharmacological reduction of TIA1 activity may promote normalization of tau:microtubule interaction and potentially ameliorate disease-related symptomatology in the PS19 model.

### Experiment 4: Drug-Induced Rescue of Cellular Pathology in rTg4510 Mouse Model (FIG. 7)

rTg4510 transgenic mice are a well-established model of tauopathy based on expression of a human tau mutant (P301L). These mice exhibit a well-defined chronopathology that includes the development of neuroinflammation (as indicated by staining with GFAP and other related markers) in the hippocampus and cortex, as well as neuronal loss. Given that these cellular pathological events are driven by tau, TIA1 antagonists compounds should limit tau-dependent pathology.

rTg4510 transgenic mice were treated with DMSO vehicle or OTX1017 (Formula I in claims 5 mg/kg i.p.) once per week for 7 weeks beginning at 77 d. Hippocampal slices were then prepared for conventional immunohistochemistry with GFAP (neuroinflammation marker) and NeuN (neuronal marker), followed by quantitation using NIH ImageJ. GFAP signal was quantitated in cortex (ctx) (white arrows). NeuN staining was used to calculate average thickness of the CA1 pyramidal cell layer for each slice.

Compared to noncarrier control mice, rTg4510 mice exhibit significantly higher GFAP staining in the cortex, which in turn is ameliorated by administration of OTX1017 (Formula I in claims) (FIG. 7). The thickness of the CA1 pyramidal layer neurons, which reflects neuronal number and cell body volume, is reduced in the transgenic background, but ameliorated by drug treatment (FIG. 7, blue arrows).

Chronic treatment of transgenic mice with OTX1017 (Formula I in claims) reduces neuroinflammation, as judged by GFAP staining, and confers a significant degree of neuroprotection to hippocampal layer neurons, as demonstrated by maintenance of NeuN staining. Phenotypic rescue by OTX1017 (Formula I in claims) should be associated with a reduction in TIA1-mediated generation of toxic tau oligomers in this transgenic model. These results support the therapeutic potential of OTX1017 (Formula I in claims) and related chemical structures in the context of tauopathies.

### Experiment 5: TIA1 Antagonists Block Arsenite-Induced Stress Granule Formation and Prevent Recruitment of Tau Protein into Stress Granules (FIG. 8)

Having identified compounds that bind to TIA1 and induce multimerization in vitro, the effect of such compounds on the formation of stress granules in cell lines was determined, given that TIA1 is a nucleator of stress granules. Furthermore, given that tau is a component of endogenous stress granules, it is important to determine whether TIA1 antagonists impact recruitment of tau into stress granules.

Sodium arsenite (As) is a potent oxidative stressor that consistently induces TIA1-positive stress granules in many different cell lines. In this representative experiment, HT22 cells were treated concurrently with As (0.5 mM) and TIA1 antagonist (100 μM) for 30 min. followed by paraformaldehyde fixation and standard fluorescence immunocytochemistry to assess stress granule formation and TIA1/tau colocalization into stress granules. Images were quantitated using NIH ImageJ particle analysis.

Sodium arsenite consistently induces stress granule formation in virtually all cells in the treated group (FIG. 8, arrows indicate endogenous TIA1- and tau-positive stress granules). Coincubation of arsenite with Ref49 (also known as OTX0049) and OTX1035 (a drug of the same chemical scaffold) shows drug-dependent inhibition of arsenite-induced stress granule formation (FIG. 8, images and first graph). Quantitation of tau foci indicates drug-dependent reduction in the average size of arsenite-induced tau foci (FIG. 8, right graphs).

These representative data indicate that TIA1 compounds identified from the in vitro FRET screen inhibit stress granule formation in a cell culture-based assay. While the results from the experiments may initially appear paradoxical (i.e., the drugs cause TIA1 multimerization in the FRET assay but block stress granule formation in cells), they are consistent with the idea that the compounds bind TIA1 and enforce a structural configuration that precludes recruitment into stress granules. By targeting TIA1, the compounds also prevent recruitment of other stress granule components such as tau.

### Experiment 6: TIA1 Antagonists Block Puromycin-Induced Stress Granules in Human Motor Neurons (FIG. 9)

Experiment 6 establishes that TIA1 antagonists inhibit arsenite-induced stress granule formation in HT22 cells, a murine cell line. Here, determine that TIA1 antagonists can likewise inhibit puromycin-induced stress granules in human C9-ALS patient-derived motor neurons.

iPSC-derived motor neurons were generated from human ALS patients by the Stem Cell Core facility at Columbia University. Motor neurons were then treated for 24 h with puromycin (5 μg/ml), a protein synthesis inhibitor that induces stress granules in a variety of cell types, along with either DMSO (vehicle) or a TIA1 antagonist as indicated. Standard fixation with paraformaldehyde followed by immunocytochemistry using an antibody against G3BP, a definitive stress granule component, or TIA1 itself, was performed, along with Hoechst staining to label nuclei. Images were acquired by standard confocal microscopy, followed by quantitation of G3BP- or TIA1-positive foci per cell.

Puromycin induces stress granules in virtually all motor neurons in vehicle-treated samples (FIG. 9A, arrows), as shown in these representative images. In contrast, co-incubation of puromycin with OTX0049 at indicated concentrations completely inhibits stress granule formation (FIG. 9, top panel). Drug-dependent inhibition of TIA1-positive stress granules is evident across a variety of human ALS patient-derived motor neuron lines (FIG. 9, bottom panel).

With studies performed using two different stressors (arsenite and puromycin) and distinct cell types (mouse cell line as well as human motor neurons from normal individuals or ALS patients), our data are consistent with the idea that TIA1 is a key nucleator of stress granules in a variety of experimental contexts. The results demonstrate that stress granules can be effectively targeted using TIA1 antagonists identified in the in vitro FRET screen.

The disclosures of each patent, patent application, and publication cited or described in this document are hereby incorporated herein by reference, in its entirety.

Those skilled in the art will appreciate that numerous changes and modifications can be made to the preferred embodiments of the methods, compounds, and compositions disclosed and that such changes and modifications can be made without departing from the spirit of the methods, compounds, and compositions. It is, therefore, intended that the appended claims cover all such equivalent variations as fall within the true spirit and scope of the methods, compounds, and compositions.

### Embodiments

The following list of embodiments is intended to complement, rather than displace or supersede, the previous descriptions.

Embodiment 1. A method of treating and/or prophylaxis of a disease or condition associated with cellular stress granule formation in a mammal, the method comprising:
administering one or more of the compounds of Formulas (I) to (IV), or a pharmaceutically acceptable salt thereof:

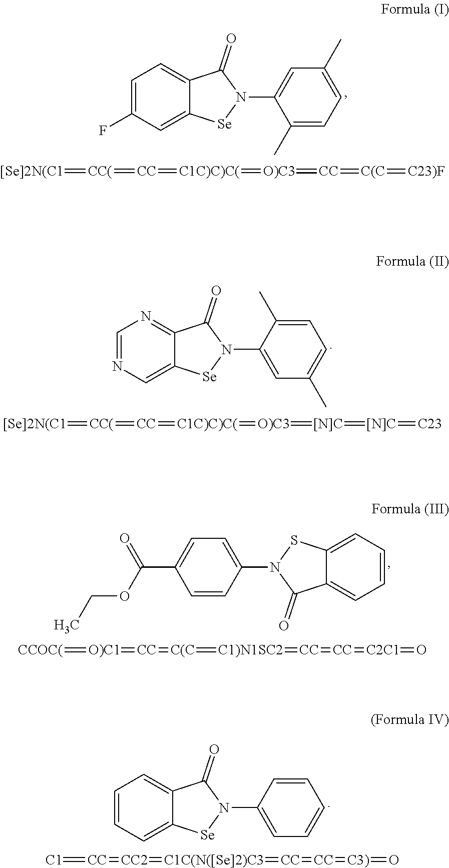

Embodiment 2. The method of embodiment 1, wherein the cellular stress granules comprise TIA1, Tau, TDP-43, or a combination thereof.

Embodiment 3. The method of any one of embodiments 1 or 2, wherein the disease or condition is a neurodegenerative disorder, a viral infection, a disease-linked genetic mutation, Welander distal myopathy, chronic stress, aging, psychiatric illness, or cancer.

Embodiment 4. The method of embodiment 3, wherein the neurodegenerative disorder is amyotrophic lateral sclerosis (ALS), frontotemporal dementia (FTD), Alzheimer's disease (AD), and tauopathies.

Embodiment 5. The method of embodiment 3, wherein the psychiatric illness is post-traumatic stress disorder (PTSD) or anxiety.

Embodiment 6. The method of embodiment 3, wherein the cancer is associated with a KRAS mutation.

Embodiment 7. The method of any one of the previous embodiments, wherein administration of the one or more compounds promotes multimerization of TIA1.

Embodiment 8. The method of any one of the previous embodiments, wherein administration of the one or more compounds prevents or decreases cellular stress granules or the formation thereof.

Embodiment 9. The method of any one of the previous embodiments, wherein administration of the one or more compounds prevents or decreases Tau or TDP-43 aggregation or oligomerization.

Embodiment 10. The method of any one of the previous embodiments, further comprising administering chemotherapeutic drugs to the mammal.

Embodiment 11. The method of embodiment 10, wherein the chemotherapeutic drug is sorafenib or bortezomib.

Embodiment 12. The method of any one of the previous embodiments, wherein following the administering the compound is delivered to a neuron.

Embodiment 13. The method of any one of the previous embodiments, wherein the mammal is a human.

Embodiment 14. A compound according to any one of Formula (I), Formula (II), or a pharmaceutically acceptable salt thereof:

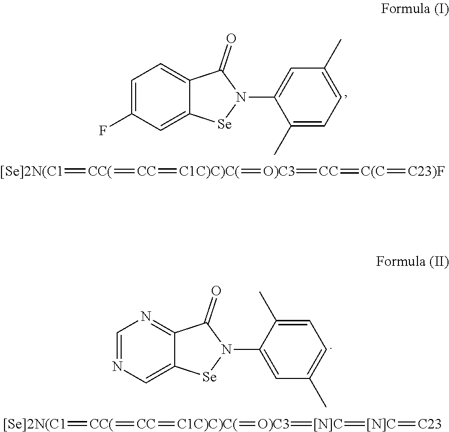

Embodiment 15. A composition, comprising:
one or more compounds according to Formula (I), Formula (II), or a pharmaceutically acceptable salt, solvate, hydrate or tautomer thereof:

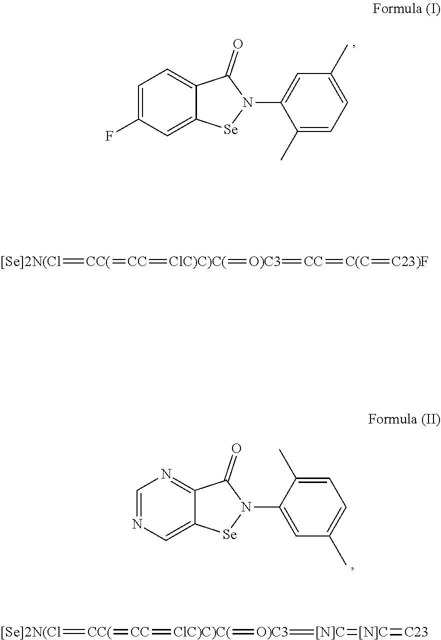

Embodiment 16. A method of identifying agents that prevent and/or reduce cellular stress granule formation comprising:
a.) incubating a TIA1 protein or a TIA1 fusion protein with one or more agents; and
b.) detecting the dimerization, oligomerization, or other multimeric state of the TIA1 protein or the TIA1 fusion protein, wherein the presence of multimeric TIA1 or multimeric TIA1 fusion protein indicates that the agent is capable of preventing and/or reducing cellular stress granule formation.

Embodiment 17. The method of embodiment 16, wherein the presence of multimeric TIA1 is detected via Förster resonance energy transfer (FRET).

Embodiment 18. The method according to embodiments 16 or 17, wherein the agent is a small molecule.

Embodiment 19. The method according to embodiments 16-18, wherein the fusion protein comprises TIA1 and a fluorescent protein.

What is claimed:

1. A method of treating and/or prophylaxis of a disease or condition associated with cellular stress granule formation in a mammal, the method comprising:
administering one or more of the compounds of Formulas (I) to (IV), or a pharmaceutically acceptable salt thereof:

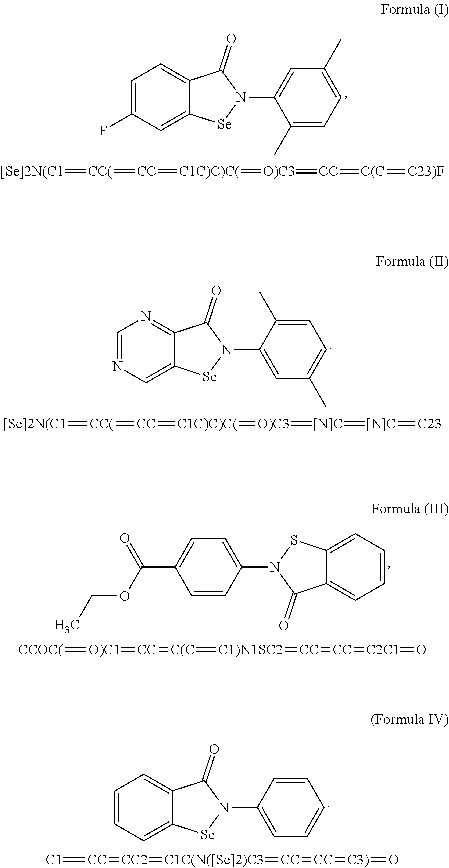

2. The method of claim 1, wherein the cellular stress granules comprise TIA1, Tau, TDP-43, or a combination thereof.

3. The method of claim 1, wherein the disease or condition is a neurodegenerative disorder, a viral infection, a disease-linked genetic mutation, Welander distal myopathy, chronic stress, aging, psychiatric illness, or cancer.

4. The method of claim 3, wherein the neurodegenerative disorder is amyotrophic lateral sclerosis (ALS), frontotemporal dementia (FTD), Alzheimer's disease (AD), or tauopathies.

5. The method of claim 3, wherein the psychiatric illness is post-traumatic stress disorder (PTSD) or anxiety.

6. The method of claim 3, wherein the cancer is associated with a KRAS mutation.

7. The method of claim 1, wherein administration of the one or more compounds promotes multimerization of TIA1.

8. The method of claim 1, wherein administration of the one or more compounds prevents or decreases cellular stress granules or the formation thereof.

9. The method of claim 1, wherein administration of the one or more compounds prevents or decreases Tau or TDP-43 aggregation or oligomerization.

10. The method of claim 1, further comprising administering chemotherapeutic drugs to the mammal.

11. The method of claim 10, wherein the chemotherapeutic drug is sorafenib or bortezomib.

12. The method of claim 1, wherein following the administering the compound is delivered to a neuron.

13. The method of claim 1, wherein the mammal is a human.

14. A compound according to any one of Formula (I), Formula (II), or a pharmaceutically acceptable salt thereof:

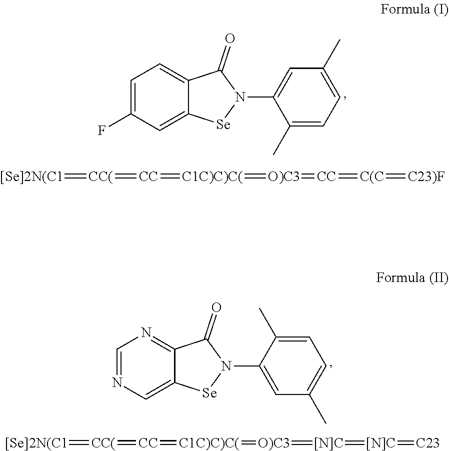

15. A composition, comprising:
one or more compounds according to Formula (1), Formula (II), or a pharmaceutically acceptable salt, solvate, hydrate or tautomer thereof:

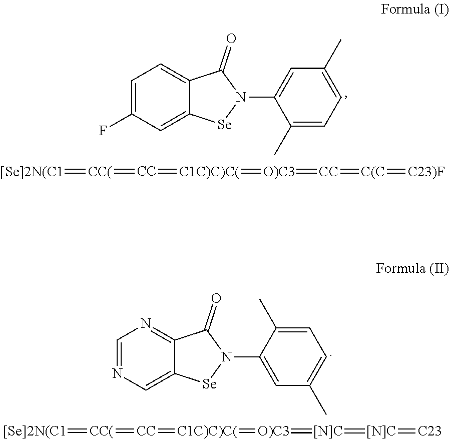

16. A method of identifying agents that prevent and/or reduce cellular stress granule formation comprising:
a.) incubating a TIA1 protein or a TIA1 fusion protein with one or more agents; and
b.) detecting the dimerization, oligomerization, or other multimeric state of the TIA1 protein or the TIA1 fusion protein, wherein the presence of multimeric TIA1 or multimeric TIA1 fusion protein indicates that the agent is capable of preventing and/or reducing cellular stress granule formation.

17. The method of claim 16, wherein the presence of multimeric TIA1 is detected via Förster resonance energy transfer (FRET).

18. The method of claim 16, wherein the agent is a small molecule.

19. The method of claim 16, wherein the fusion protein comprises TIA1 and a fluorescent protein.
